(19) United States
(12) Patent Application Publication
(10) Pub. No.: US 2006/0008380 A1
(43) Pub. Date: Jan. 12, 2006

(54) Apparatus and method for reduction of gas microbubbles

(76) Inventors: Alexei Moozyckine, Sketty (GB); John Dingley, Llangefellach (GB)

Correspondence Address: CONLEY ROSE, P.C., P. O. BOX 3267, HOUSTON, TX, 77253-3267, US

(21) Appl. No.: 11/101,794

(22) Filed: Apr. 8, 2005

Related U.S. Application Data
(63) Continuation-in-part of application No. PCT/GB03/04425, filed on Oct. 13, 2003.

(30) Foreign Application Priority Data
Oct. 11, 2002 (GB) 0223577.8
Oct. 11, 2002 (GB) 0223578.6

(51) Int. Cl. A61M 1/14; A61M 37/00; A61M 1/34

(52) U.S. Cl. 422/45; 604/4.01

(57) ABSTRACT

A method and corresponding apparatus for removing microbubbles from blood incorporating a device through which the blood passes in a continuous linear flow; devices which can be used are a magnetic device and/or a Venturi device.

This application is a continuation-in-part of application PCT/GB2003/004425, filed Oct. 13, 2003, which claimed priority to Great Britain Application No. 0223577.8, filed Oct. 11, 2002, and Great Britain Application No. 0223578.6, filed Oct. 11, 2002. Each of the above-listed applications is hereby incorporated in their entirety herein by reference for all purposes.

The present invention relates to a method and a device for reducing gas microbubbles in fluids, particularly it relates to a method and device for the reduction of gas microbubbles formed in the bloodstream during the use of a cardiopulmonary bypass circuit.

The device is designed to be used with any standard cardiopulmonary bypass circuit and is aimed at reducing the amount of microbubbles in blood formed at any stage of the blood circuit, and ideally could be installed in the position prior to the cannula entrance.

It is well documented that gas microbubbles produced during cardiopulmonary bypass are predominantly responsible for serious postoperative psycho-neurological dysfunction. (Refs. 1-10). At present, neuropsychologic impairment, to which intraoperative cerebral microemboli are a principal cause, is the most common complication of coronary bypass surgery. (Refs. 1, 2, 4-10).

In numerous clinical studies of this phenomenon, Doppler ultrasonography (Refs. 1, 3-5, 7, 9, 11, 12) was used to detect the number of microemboli in the cerebral arteries of patients. It was found that these emboli are air microbubbles that are not eliminated by the arterial line filter, and further attempts have been (Refs. 7, 11) to reduce the amount of these microbubbles using various traps.

One of the latest developments (Ref. 7) includes a dynamic bubble trap, placed in the arterial line between the arterial filter and arterial cannula, where the bloodstream is forced to rotate and bubbles are driven by centripetal force to the centre of the axial blood flow, where they are collected and returned to the cardiotomy reservoir. This design (Ref. 7) allows for a significant reduction of microbubbles in the arterial line, and as a consequence, a decrease of high-intensity transient signals in the brain of patients was observed.

We have now devised new methods and devices which can be used for the reduction of microbubbles in blood. The devices can be used with any standard cardiopulmonary bypass circuit to reduce the amount of microbubbles in blood formed at any stage of the blood circuit prior to the cannula entrance.

According to the invention there is provided a method for reducing microbubbles in blood or other biological fluid which method comprises passing the blood in a linear flow through a device in which the microbubbles separate from the blood or other biological fluid.

By linear is meant that the flow is substantially in one direction through the device.

The flow is preferably substantially laminar through the device.

This is different to the device of Ref. 7 where the bloodstream is forced to rotate and bubbles are driven by centripetal force to the centre of the axial blood flow and the flow through the device is not continuous.

One device which can be used in the present invention is a magnetic device and in one embodiment of the invention there is provided a method for reducing microbubbles in blood which method comprises passing the blood through a magnetic field formed by a magnet or magnets.

The invention also provides apparatus or a device for removing microbubbles from blood which comprises a device comprising (i) one or several/many conduit(s) down which blood can be passed and (ii) a means for generating a magnetic field(s) positioned so that blood flowing down the conduit(s) passes through the magnetic field(s).

The device can be used in vivo in a situation where blood is passed from a body (human or animal) through the device of the invention and then back to the body for example in conjunction with any of the existing blood circuits e.g. the device can be used with any standard cardiopulmonary bypass circuit, in order to reduce the amount of gas microemboli in the bloodstream formed during cardiopulmonary bypass surgery, thus minimizing subsequent brain injury in cardiopulmonary surgery. In a typical circuit the blood is pumped through a blood reservoir, an oxygenator, a filter and/or bubble trap and back to the body. Usually there are regulators and flow meters to control the rate of flow etc. The invention is preferably located in the circuit after the excess of air/gas has been removed from the bloodstream using bubble traps or other filters.

It is thought that the invention works by solubilizing the remaining gas microbubbles in the blood and thus removing the risk of their interaction with and accumulation into the brain tissues/capillaries.

In use the magnetic treatment device comprises magnets (permanent or electro magnets) which can be located round the conduit(s) such as pipe(s) e.g. by clamping to the pipe or by having the magnets positioned around the outside of a pipe, so the pipe flows through a central magnetic field. The pipe can be any conventional pipework used in cardiopulmonary circuits. The magnetic fields of the magnet or magnets can be made very strong if necessary by the use of so called “super magnets” made of strongly ferromagnetic alloys.

All other devices used to reduce microemboli trauma (arterial filters, bubble traps) are aimed at the reduction of the amount of gas microbubbles formed at any stage of cardiopulmonary bypass apart from the very last one (arterial cannulation), determined only by the design, geometry and material of the aortic cannula, (Ref. 9) the magnetic device of the present invention is capable of reducing gas embolism even at this last stage by altering the surface tension of blood and thus preventing/reducing the formation of microbubbles at all stages of the heart bypass blood circuitry.

Magnetic treatment devices (MTDs) have been used for treating water. In a simple case, the water passes through the applied magnetic field created by a permanent magnet or electromagnet (or a combination of these). Despite extensive controversy over the nature of water magnetic effect and even the existence of the effect itself (probably, resulting from the relatively subtle nature of the effect and a variety of conditions used by different researchers), at present there exists a convincing body of articles in various journals (Refs. 13-63) documenting the studies of the long-term electromagnetic radiation effect on fluids and its practical applications.

At present, MTDs commercially available for antiscale water treatment are relatively inexpensive and compact kits, available commercially from several manufacturers in the UK, US, Germany, Denmark and other countries.

In recent years the nature of magnetic water conditioning phenomenon has been studied using a variety of techniques and currently is thought to be attributed to water-air (water-gas) interface effects. Submicroscopic gas bubbles and clusters thereof in water of approximate diameter 1-10 nm, probably stabilized by ions, are considered to be responsible for so-called “magnetic water memory” effect. This effect reveals itself in several recordable changes of water properties, including stabilization of the solution pH, changes/oscillations in conductivity and ζ (zeta) potential of colloids, reduction of metals corrosion and scale formation, inactivation of micro-organisms, enhancement of calcium efflux through biomembranes, reduction of surface tension, fluorescence of hydrophobic and hydrophilic probes, etc. It has been shown that the magnetic treatment induces changes in the crystal structure of the precipitate formed in concentrated carbonate solutions, producing mainly aragonite instead of calcite, affecting the nucleation and crystal growth. (Ref. 39).

The effect of “water magnetic memory” is long-lasting, changes in the observed properties remain for several minutes to several hours after the water has been treated by radiofrequency (RF) radiation, microwaves, magnets or electromagnets. (Refs. 13, 14, 17, 31, 50). Usually, the magnetic treatment does not require very strong RF sources or powerful magnets, although the amplitude of the applied RF field influences the observed effects. It was observed that the “water magnetic memory” effect disappears after the liquid has been carefully outgassed, this prompted the researchers to suggest (Refs. 14, 17, 31, 59) that it is the perturbation of the liquid-gas interface resulting in formation of nanobubbles (Ref 13) (and not the presence of trace concentrations of Fe²⁺ ions, as was thought before (Ref. 15) that is responsible for the appearance of long-lasting effects which require hours to relax.

It has been found recently (Refs. 45-49) that magnetic water treatment also increases the chlorine retention by swimming pool water, suppressing free chlorine loss and thus inhibiting microbial growth.

Apparently, the main driving force behind the decrease in chlorine desorption can be found in altered surface tension of the solution, after its exposure to the electromagnetic field, followed by changes in the solubility of the gas. We propose to apply the same effect to help solubilize the remaining oxygen (or air, or any other gas, or a mixture of thereof) microemboli in the blood, and thus reduce their effervescence in brain capillaries.

In general, the device containing a set of permanent or electromagnets is clamped to/around the fluid pipe made of metal, plastic or any other material. The MTD does not require direct contact with the fluid, which is particularly important for artificial blood circulation systems, in order to minimize the allergic reactions of the body.

It is a feature of the present invention that the implementation of the magnetic device(s) of the present invention involves minimum expenditure (as MTDs can be easily incorporated into existing cardiopulmonary bypass equipment) and, once assembled, do not require any special attention from the medical personnel.

Also, magnetic devices are extremely cost-effective and do not require sterilization as they do not work in direct contact with blood and do not have any moving parts, so their lifetime is restricted only by the durability of the materials used for their clamps.

In another embodiment of the invention the device for the reduction of gas microbubbles in blood comprises a Venturi tube formed of a first and second truncated cones connected together at their narrower ends, with an inlet for blood at the wider end of the first truncated cone and an outlet for blood at the wider end of the second truncated cone.

The invention also provides a method for treating blood which comprises passing the blood through a Venturi device(s) which comprises one or several Venturi tube(s) formed of a first and second truncated cone connected together at their narrower ends with an inlet for blood at the wider end of the first truncated cone and an outlet for blood at the wider end of the second truncated cone.

Preferably there is a connecting tube connecting the narrow ends of the first and second truncated cones, called the throat.

The interior sides of the first and second truncated cones are preferably linear with a substantially constant angle of taper, although curved sides and varying angles of taper can be used, as in conventional Venturi devices. Preferably the interior surfaces of the truncated cones are smooth to facilitate laminar flow.

Venturi tubes are frequently used in hydraulic engineering for the measurement of flow rates (Refs. 13-22). A usual design of the Venturi tube (Refs. 13-18, 21, 22) includes two truncated cones (inlet and outlet) connected together by a short cylindrical pipe of a smaller diameter, called the throat and usually installed horizontally.

When the blood is pumped through the Venturi device its velocity will increase as it passes down the first truncated cone which will reduce the pressure according to the modified Bernoulli's equation:

$$h = \frac{{P}_{1} - {P}_{2}}{γ} = \frac{{v}_{2}^{2} - {v}_{1}^{2}}{2 g} \qquad ( I )$$

where γ represents specific weight of the fluid, P₁, P₂ and ν₁, ν₂ represent pressure and velocity of the fluid in sections 1 and 2 corresponding to diameters D of the pipe and d of the throat, so that ν₂/ν₁=D/d.

A ratio D/d around 4 or less is preferred as this produces a considerably low pressure at the throat, sufficient to cause liberation of the dissolved air/gas, (Refs. 14, 21, 23, 24).

The ratio D/d is limited for a given flow rate and temperature by the maximum allowed pressure drop in the throat; for too high ratios, the velocity of the fluid at the throat can be very high, and the resulting pressure drop too big, capable of producing a subatmospheric pressure (known as a Venturi vacuum, Ref. 21) and vaporization of the liquid at this point. (Ref. 14). This phenomenon, called cavitation, is a highly undesired event, (Ref. 25) as it can cause severe damage to the blood cells, therefore the D/d ratio should always be well below the cavitation threshold.

The converging section of the first truncated cone (upstream from the throat) preferably has a gradient (inclination to the longitudinal axis or half angle) 10-30 degrees, the diverging section (downstream from the throat) preferably has a gradient 2.5-14 degrees. A long cone/form modification of the Venturi tube, rather than a short cone one, can be more suitable for medical applications, as it has lower pressure loss (Ref. 17) and creates less turbulence to the fluid flow, thus minimizing the potential damage to the blood cells.

In the present invention preferably the device is positioned so that excess of the dissolved oxygen (or any other gas) is evolved from the blood prior to administration of the oxygenated blood to the patient.

Obviously, this procedure can only reduce the amount of oxygen dissolved in blood and does not affect in any way the amount of oxygen chemically bound to haemoglobin (neglecting very small changes in equilibrium constant), and thus does not change the uptake of aidful oxygen by the blood. Smooth laminar flow inside the Venturi tubes does not cause haemolysis of erythrocytes and therefore does not reduce the uptake of oxygen even indirectly

The device of the present invention can be used for the reduction of microbubbles in blood and can be used in conjunction with any of the bubble traps and filters, which allows to improve the efficiency of the removal of gas microemboli from the bloodstream during cardiopulmonary bypass and to reduce subsequent brain injury.

In use a gradual pressure growth in the second, diverging, truncated cone cannot quickly dissolve back the bubbles that were formed and released in the throat of the Venturi tube, (Ref. 23) so they are carried with the blood flow into a separating device or a blood filter installed downstream. The diameter of the outlet of the second truncated cone is preferably similar or larger than that of the inlet of the first truncated cone, in order to sustain a relatively slow fluid flow and to help the evolved gas to separate.

Optionally there can be a separating chamber positioned close to (or combined with) the outlet of the second truncated cone with an incorporated mesh (or several meshes) installed at an angle, β less than 90° to the direction of the flow. When the chamber is positioned horizontally, the bubbles, comparable or larger than the mesh size, travel slowly along the mesh and up to the top part of the chamber. From there, a small portion of blood, saturated with bubbles, is redirected back to the inlet of the blood pump via a bypass. The flow rate in the bypass can be regulated by a valve or clamp in order to obtain a desirable ratio of volumetric rates in the bypass and the main line (e.g. around 1/10).

The blood flow, instead of passing through the reclined mesh, can be directed into a short spiral tube or other device where the fluid is forced to rotate in order to allow the centripetal force to separate the bubbles. Alternatively, in the cardiopulmonary circuit installation, the device of the present invention can be immediately followed by the dynamic bubble trap (Ref. 7) or any other conventional blood filter; in this case no special separation chamber need be incorporated into the device's design.

The design of the separating chamber is not relevant to the present invention as a separating chamber is needed only to separate the bubbles that have been evolved in the Venturi tubes.

The advantages of using the Venturi tube (Refs. 13, 17) over other devices include its ability to sustain relatively high flow rates, very small unrecovered pressure loss, h_{L}, normally less than 12-15% of differential pressure, h, and, above all, the fluid flow through the Venturi tube is smooth, without creating a turbulence. This latter point is very important, as blood cell damage and particularly haemolysis of red blood cells represents one of the most serious negative effects during cardiac surgery, (Ref. 9) and is thought to be caused by mechanical damage induced by the compulsory circulation, oxygenation, etc.

The Venturi tube can be installed vertically as the downward flow might be more effective than the conventional horizontal mode, as formed gas bubbles spend more time in the low pressure (throat) region due to their buoyancy (Ref. 26) and have more time to grow to a size large enough to be readily separated. Also, vertical positioning of the Venturi tube reduces the area used, making the equipment more compact and better adjusted to clinical conditions.

In the specification vertically and horizontally with regard to the Venturi device means that the axis of the first and second truncated cones are vertical or horizontal respectively (although not necessarily coaxial) and so the axis of the throat is vertical or horizontal.

It is a feature of the present invention that the Venturi tube device is relatively inexpensive and can be made/assembled from any suitable materials that are adequate for handling blood, e.g. titanium or surgical stainless steel with or without coating, polymers, composites, etc.

The Venturi tube device can be used to reduce the amount of gas microemboli formed at any stage of cardiopulmonary bypass by reducing the amount of physically dissolved gas in blood, thus preventing this gas from evolving. The dissolved gas may be oxygen, xenon, or any other gas or gas mixture. The implementation of the Venturi device involves minimum expenditure as it can be easily incorporated into existing cardiopulmonary bypass equipment and does not require any special attention from the medical personnel. Also, the device is very simple to produce, cost-effective and reliable as it does not have any moving parts.

The invention is illustrated in the accompanying drawings in which:—

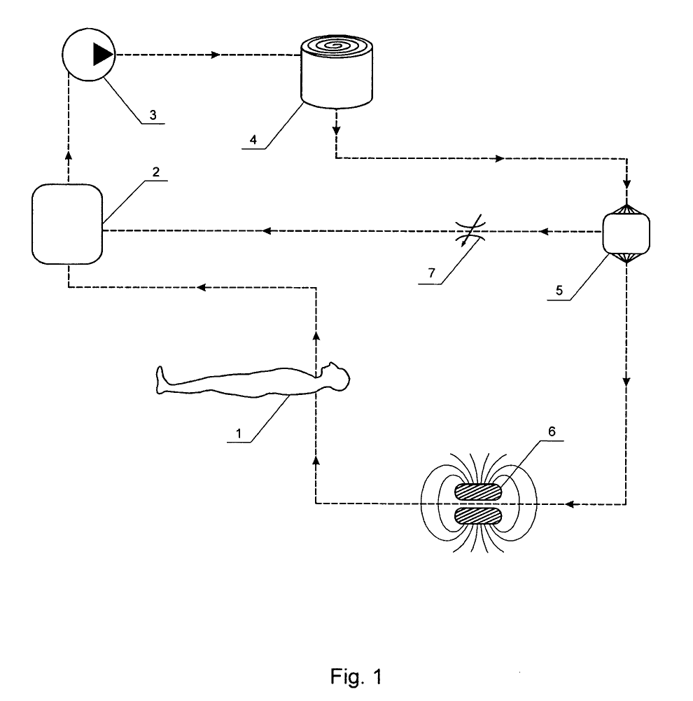

FIG. 1 shows schematically a circuit for treating blood using a magnetic device

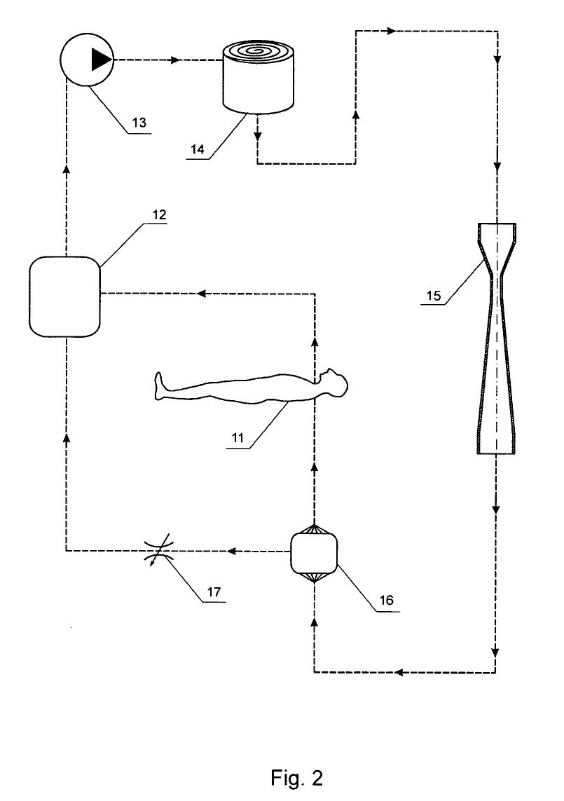

FIG. 2 shows a schematic view of the circuit incorporating a Venturi device and

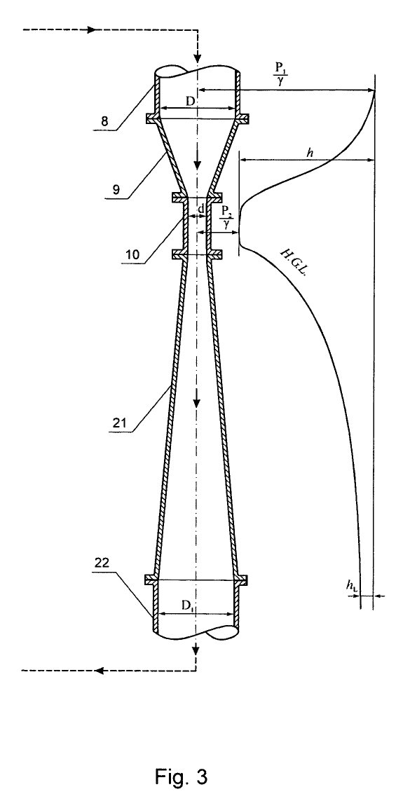

FIG. 3 is a sectional view through the Venturi device of FIG. 2.

Referring to FIG. 1, a cardiopulmonary circuit comprises a blood pump (2), a blood reservoir (3), oxygenator (4), filter (5), a magnetic treatment device (6) for use with patient shown as (1). The magnetic treatment device consisted of a tube to the outside of which are clamped permanent magnets so that blood flowing through the tube passes through the magnetic field.

In use blood from patient (1) is pumped around the circuit as shown by the arrows as in conventional cardiopulmonary circuits. When the blood passes through the device (6) before being returned to the body, the magnetic field of the device helps to solubilize any microbubbles in the blood.

Referring to FIG. 2, a cardiopulmonary circuit is shown in which there is a patient (11) from whom blood is pumped by pump (13) through reservoir (12), oxygenator (14), Venturi device (15), filter (16) back to patient (11); there is regulating valve etc. at (17).

Referring to FIG. 3, the Venturi device comprises a first truncated cone (9) which has an inlet (8) of diameter ‘D’, the outlet of the cone (9) is connected to tube (10) of diameter ‘d’. The outlet of tube (10) connects to the inlet of truncated cone (21). There is outlet (22) of truncated cone (21) which has a diameter ‘D₁’.

In use, after the blood pump and optionally a small blood settling reservoir (not shown) the blood enters the inlet (8) of diameter ‘D’ and passes down first truncated cone Venturi tube (9) through its narrow part (throat) (10) of diameter ‘d’, where the velocity of blood significantly increases and, according to the Bernoulli formula (I), the pressure drops sharply, the blood then flows down the second truncated cone (21) and out through outlet (22) of diameter D₁. This pressure drop allows some microbubbles that were previously dissolved in the blood to grow rapidly, effervesce and to be eliminated from the bloodstream. The line HGL refers to Hydraulic Grade Line, (Refs. 13, 14) otherwise known as hydraulic gradient (Ref. 22) and reflects static pressure in the system; h is the differential pressure, and h_{L} is the unrecovered pressure loss.

### REFERENCES

1. R. L. Taylor, M. A. Borger, R. D. Weisel, L. Fedorko, C. M. Feindel, Ann. Thorac. Surg., 1999, 68, 89-93.
2. M. A. Borger, C. M. Peniston, R. D. Weisel, M. Vasiliou, R. E. A. Green, C. M. Feindel, J. Thorac. Cardiov. Sur., 2001, 121, 743-749.
3. S. M. F. Malheiros, A. R. Massaro, A. A. Gabbai, C. J. N. Pessa, L. R. Gerola, J. N. R. Branco, E. B. Lira, D. M J. Christofalo, D. Federico, A. C. Carvalho, E. Buffalo, Arq. Neuropsiquiat., 2001, 59, 1-5.
4. F. Schneider, J. F. Onnasch, V. Falk, T. Walther, R. Autschbach, F. W. Mohr, Ann. Thorac. Surg., 2000, 70, 1094-1097.
5. M. A. Borger, R. L. Taylor, R. D. Weisel, G. Kulkarni, M. Benaroia, V. Rao, G. Cohen, L. Fedorko, C. M. Feindel, J. Thorac. Cardiov. Sur., 1999, 118, 740-745.
6. M. A. Borger, J. Ivanov, R. D. Weisel, V. Rao, C. M. Peniston, Eur. J. Cardio-Thorac., 2001, 19, 627-632.
7. M. Schonburg, P. Urbanek, G. Erhardt, B. Kraus, U. Taborski, A. Muhling, S. Hem, M. Roth, H. J. Tiedtke, W. P. Klovekom, Perfusion-UK, 2001, 16, 19-25.
8. A. B. Branger, D. M. Eckmann, J. Appl. Physiol., 1999, 87, 1287-1295.
9. G. P. Gravlee, R. F. Davis, M. Kurusz, J. R. Utley, eds., Cardiopulmonary Bypass: Principles and Practice, Lippincott Williams & Wilkins, Philadelphia, 2000, pp. 49-97, and references therein.
10. H. P. Grocott, J. für Anasthesie und Jntensivbehandlung, 2002, 2, 45-46.
11. X. M. Mueller, H. T. Tevaearai, D. Jegger, M. Austburger, M. Burki, L. K. von Segesser, Perfusion-UK, 1999, 14, 481-487.
12. S. Eitschberger, A. Henseler, B. Krasenbrink, B. Oedekoven, K. Mottaghy, Asaio J., 2001, 47, 18-24.
13. M. Colic, D. Morse, Phys. Rev. Lett., 1998, 80, 2465-2468.
14. M. Colic, D. Morse, Langmuir, 1998, 14, 783-787.
15. R. E. Herzog, Q. Shi, J. N. Patil, J. L. Katz, Langmuir, 1989, 5, 861-867.
16. L. C. Lipus, J. Krope, L. Crepinsek, J. Colloid Interface Sci., 2001, 236, 60-66.
17. M. Colic, D. Morse, J. Colloid Interface Sci., 1998, 200, 265-272.
18. K. Higashitani, H. Iseri, K. Okuhara, A. Kage, S. Hatade, J. Colloid Interface Sci., 1995, 172, 383-388.
19. L. Holysz, B. Chibowski, J. Colloid Interface Sci., 1994, 165, 243-251.
20. K. Higashitani, A. Kage, S. Kotamura, K. Imai, S. Hatade, J. Colloid Interface Sci., 1993, 156, 90-95.
21. K. Higashitani, K. Okuhara, S. Hatade, J. Colloid Interface Sci., 1992, 152, 125-131.
22. E. Chibowski, S. Gopalkrishnan, M. A. Bush, K. W. Bush, J. Colloid Interface Sci., 1990, 139, 43-54.
23. A. Goldsworthy, H. Whitney, E. Morris, Water Res., 1999, 33, 16 18-1626.
24. R. A. Barrett, S. A. Parsons, Water Res., 1998, 32, 609-612.
25. Y. Wang, A. J. Babchin, L. T. Chernyi, R. S. Chow, R. P. Sawatzky, Water Res., 1997, 31, 346-350.
26. S. A. Parsons, B.-L. Wang, S. J. Judd, T. Stephenson, Water Res., 1997, 31, 339-342.
27. J. S. Baker, S. J. Judd, Water Res., 1996, 30, 247-260.
28. R. Gehr, Z. A. Zhai, J. A. Finch, S. R. Rao, Water Res., 1995, 29, 933-940.
29. J. M. D. Coey, S. Cass, J. Magnet. Magnet. Mater., 2000, 209, 7 1-74.
30. J. J. Lin, J. Yotvat, J. Magnet. Magnet. Mater., 1990, 83, 525-526.
31. M. Colic, D. Morse, Colloids Surfaces A, 1999, 154, 167-174.
32. L. Yezek, R. L. Rowell, M. Larwa, E. Chibowski, Colloids Surfaces A, 1998, 141, 67-72.
33. K. Higashitani, J. Oshitani, N. Obmura, Colloids Surfaces A, 1996, 109, 167-173.
34. E. Chibowski, L. Holysz, Colloids Surfaces A, 1995, 101, 99-101.
35. E. Chibowski, L. Holysz, W. Wójcik, Colloids Surfaces A, 1994, 92, 79-85.
36. N. Su, Y.-H. Wu, C.-Y. Mar, Cement Concrete Res., 2000, 30, 599-605.
37. A. Chiba, W.-C. Wu, A. Terashita, J. Mater. Sci., 1996, 31, 3821-3825.
38. E. Chibowski, L. Holysz, M. Lubomska, J. Adhesion Sci. Technol., 1999, 13, 1103-1117.
39. J. S. Baker, S. J. Judd, S. A. Parsons, Desalination, 1997, 110, 15 1-165.
40. K. W. Busch, M. A. Busch, Desalination, 1997, 109, 131-148.
41. N. Rocha, G. Gonzalez, L. C. doC. Marques, D. S. Vaitsman, Petrol. Sci. Technol., 2000, 18, 33-50.
42. Y. Zhao, L. Zhao, X. Wei, B. Han, H. Yan, J. Therm. Anal., 1995, 45, 13-16.
43. F. Y. Ishihara, S. M. Bradley, J. Imaging Technol., 1988, 14, 157-160.
44. J. R. Newman, R. C. Watson, Hydrobiobogia, 1999, 415, 319-322.
45. J. E. Burgess, S. J. Judd, S. A. Parsons, Process Safety Environ. Prot., 2000, 78, 213-218.
46. K. Higashitani, J. Oshitani, Process Safety Environ. Prot., 1997, 75, 115-119.
47. K. W. Busch, M. A. Busch, R. E. Darling, S. Maggard, S. W. Kubala, Process Safety Environ. Prot., 1997, 75, 105-1 14.
48. S. A. Parsons, S. J. Judd, T. Stephenson, S. Udol, B.-L. Wang, Process Safety Environ. Prot., 1997, 75, 98-104.
49. A. S. Ifill, J. S. Baker. S. J. Judd, Process Safety Environ. Prot., 1996, 74, 120-124.
50. M. \check{C}olić, A. Chien, D. Morse, Croatica Chem. Acta, 1998, 71, 905-916.
51. R. F. Benson, R. Lubosco, D. F. Martin, J. Envron. Sci. Health A, 2000, 35. 1527-1540.
52. S. Kobe, G. Dra\check{z}ić, P. J. McGuiness, J. Stra\check{z}i\check{s}ar, Acta Chim. Slov., 2001, 48, 77-86.
53. L. Lipus, J. Krope, L. Garbai, Hung. J. Ind. Chem., 1998, 26, 109-112.
54. L. Lipus, J. Krope, L. Garbai, Hung. J. Ind. Chem., 1994, 22, 239-242.
55. E. E. Fesenko, A. Y. Gluvstein, FEBS Lett., 1995, 367, 53-55.
56. E. E. Fesenko, V.1. Geletyuk, V. N. Kazachenko, N. K. Chemeris, FEBS Lett., 1995, 366, 49-52.
57. V.1. Geletyuk, V. N. Kazachenko, N. K. Chemeris, E. E. Fesenko, FEBS Lett., 1995, 359, 85-88.
58. C. Gabrielli, R. Jaouhari, G. Maurin, M. Keddam, Water Res., 2001, 35, 3249-3259.
59. M. Lubomska, B. Chibowski, Langmuir, 2001, 17, 4181-4188.
60. J. Oshitani, R. Uehara, K. Higashitani, J. Colloid Interface Sci., 1999, 209, 374-379.
61. J. Oshitani, D. Yamada, M. Miyahara, K. Higashitani, J. Colloid Interface Sci., 1999, 210, 1-7.
62. M. Morimitsu, K. Shiomi, M. Matsunaga, J. Colloid Interface Sci., 2000, 229, 641-643.
63. A. Khalil, R. Rosset, C. Gabrielli, M. Keddam, H. Perrot, J. Appl. Electrochem., 1999, 29, 339-346.

1. A method for reducing microbubbles in blood or other biological fluids which method comprises passing the blood or other fluid in a linear flow through a device in which the microbubbles separate from the blood.

2. The method of claim 1 in which the flow is substantially laminar through the device.

3. An apparatus for removing microbubbles from blood which comprises (i) a conduit down which blood can be passed and (ii) a separating device through which the blood can flow in a continuous linear flow in which microbubbles separate from the blood.

4. The apparatus of claim 3 which comprises (i) a conduit down which blood can be passed and (ii) a means for generating a magnetic field positioned so that blood flowing down the conduit passes through the magnetic field.

5. The apparatus of claim 3 which comprises (i) a pump (ii) an oxygenator (iii) a filter and (iv) a magnetic treatment device in which, in use, the pump pumps blood from a body through the oxygenator and filter and then through the magnetic treatment device and back to the body and in which the magnetic treatment device comprises a conduit down which the blood flows and at least one magnet located so that the blood flows through the magnetic field generated by the magnet.

6. The apparatus of claim 3 in which there is a blood reservoir, regulators, controllers, or combinations thereof through which the blood flows to control the rate of flow of the blood.

7. The apparatus of claim 3 wherein the magnetic treatment device is located after the excess of air/gas has been removed from the bloodstream using bubble traps or other filters.

8. The apparatus of claim 3 in which the magnetic treatment device comprises at least one permanent or electromagnet.

9. A method for reducing microbubbles in blood which method comprises passing the blood in a continuous linear flow through a magnetic field formed by a magnet.

10. The method of claim 9 which comprises pumping blood from a patient and passing the blood through an oxygenator, a filter and a magnetic treatment device incorporating a conduit and a magnet so the blood flows down the conduit(s) through the magnetic field generated by the magnet(s).

11. The apparatus of claim 3 wherein the device comprises a Venturi tube formed of a first and second truncated cone connected together at their narrower ends with an inlet for blood at the wider end of the first truncated cone and an outlet for blood at the wider end of the second truncated cone.

12. The apparatus of claim 11 which comprises (i) a pump (ii) an oxygenator, (iii) a filter and the Venturi device in which, in use, the pump pumps blood from a body through the oxygenator and filter and then through the said device and back to the body.

13. The apparatus of claim 12 in which the device is positioned horizontally, vertically or at an angle.

14. The apparatus of claim 111 in which there is a connecting tube connecting the narrow ends of the first and second truncated cones.

15. The apparatus of claim 11 in which the interior sides of the first and second truncated cones in axial section are linear or curved.

16. The apparatus of claim 11 in which there is a separating means for the formed gas (micro)bubbles positioned close to, or combined with the outlet of the second truncated cone.

17. The apparatus of claim 11 which comprises (i) a pump (ii) an oxygenator, (iii) a filter and one or more Venturi devices connected in a parallel or sequential mode which, in use, the pump pumps blood from a body through the oxygenator and filter and then through the said device(s) and back to the body and in which the one or more Venturi devices are positioned so that excess of the dissolved gas or a mixture of gases is evolved from the blood prior to administration of the oxygenated blood to the patient.

18. A method for treating blood which method comprises passing the blood through a Venturi device(s) as claimed in claim 11.

19. A method for treating blood which comprises pumping the blood from a patient through a reservoir, an oxygenator, a filter, and one or more Venturi devices and back to the body and in which the one or more Venturi devices are as claimed in claim 11.

20. The apparatus of claim 17 wherein more than one Venturi device is combined in one unit.

21. The apparatus of claim 17 wherein the dissolved gas comprises oxygen, xenon, or any other gas.

22. The apparatus of claim 17 wherein the mixture of gases comprises air, an air and oxygen mixture, or an air and xenon mixture.

23. The method of claim 19 wherein the one or more Venturi devices are combined in one unit.
